(19) United States
(12) Patent Application Publication
(10) Pub. No.: US 2021/0358601 A1
(43) Pub. Date: Nov. 18, 2021

(54) ARTIFICIAL INTELLIGENCE SYSTEM FOR CLINICAL DATA SEMANTIC INTEROPERABILITY

(71) Applicant: Optum Technology, Inc., Eden Prairie, MN (US)

(72) Inventors: MURALIDHARAN RAMAMURTHY PILLAI, Hyderabad (IN); Satyajit Pati, Hyderaba (IN); Jyotiprava Pattanaik, Hyderabad (IN)

(21) Appl. No.: 15/931,167

(22) Filed: May 13, 2020

(51) Int. Cl. G16H 40/20; G06N 3/02; G06F 16/245; G16H 50/20; G16H 50/70; G16H 70/20; G16H 10/60; G06F 40/279; G06F 40/242

(52) U.S. Cl. CPC G16H 40/20; G06N 3/02; G06F 16/245; G16H 50/20; G06F 40/242; G16H 70/20; G16H 10/60; G06F 40/279; G16H 50/70

(57) ABSTRACT

Various embodiments of the present disclosure facilitate clinical data semantic interoperability using machine learning. In one example, an embodiment provides for extracting one or more medical concepts from clinical data based at least in part on a natural language processing technique, identifying corresponding clinical context for a first medical concept and a second medical concept in the clinical data by analyzing one or more medical ontologies based at least in part on an ontology traversal technique associated with a neural network, generating a score for a relationship between the first medical concept and the second medical concept based at least in part on one or more rules with respect to coded entries of the clinical data and the one or more medical ontologies associated with the ontology traversal technique, and performing one or more actions associated with the clinical data based at least in part on the score.

### BACKGROUND

The present invention addresses technical challenges related to analysis of digital data in an accurate, computationally efficient and predictively reliable manner. Existing systems are generally ill-suited to accurately, efficiently and reliably analyze and/or generate data in various domains, such as domains that are associated with high-dimensional categorical feature spaces with a high degree of size, diversity and/or cardinality.

### BRIEF SUMMARY

In general, embodiments of the present invention provide methods, apparatus, systems, computing devices, computing entities, and/or the like for analysis of digital data using artificial intelligence. Certain embodiments utilize methods, apparatus, systems, computing devices, computing entities, and/or the like for additionally providing recommendations based at least in part on the analysis of the digital data. Additionally, in certain embodiments, methods, apparatus, systems, computing devices, computing entities, and/or the like provide for a computer-based solution and/or a machine learning solution that identifies medical concepts embedded in clinical documents and segments the medical concepts into relevant medical ontologies. In certain embodiments, the computer-based solution and/or the machine learning solution contextualizes the medical concepts and derives the medical ontologies using an ontology traversal technique. In certain embodiments, a rule-based inference engine is employed to establish clinical accuracy of terms and/or phrases. In certain embodiments, a decision management system manages decisions based at least in part on accuracy scores provided by the rule-based inference engine in order to invoke actions and/or workflows for a system.

In accordance with one embodiment, a method is provided. In one embodiment, the method comprises extracting one or more medical concepts from clinical data based at least in part on a natural language processing technique. The method also comprises identifying corresponding clinical context for a first medical concept and a second medical concept in the clinical data by analyzing one or more medical ontologies based at least in part on an ontology traversal technique associated with a neural network, where the ontology traversal technique traverses an ontology knowledge graph of interlinked medical concepts with a hierarchy of class levels. The method also comprises generating a score for a relationship between the first medical concept and the second medical concept based at least in part on one or more rules with respect to coded entries of the clinical data and the one or more medical ontologies associated with the ontology traversal technique. Furthermore, the method comprises performing one or more actions associated with the clinical data based at least in part on the score for the relationship between the first medical concept and the second medical concept.

In accordance with another embodiment, an apparatus comprising at least one processor and at least one memory including computer program code is provided. In one embodiment, the at least one memory and the computer program code can be configured to, with the processor, cause the apparatus to: extract one or more medical concepts from clinical data based at least in part on a natural language processing technique, identify corresponding clinical context for a first medical concept and a second medical concept in the clinical data by analyzing one or more medical ontologies based at least in part on an ontology traversal technique associated with a neural network, generate a score for a relationship between the first medical concept and the second medical concept based at least in part on one or more rules with respect to coded entries of the clinical data and the one or more medical ontologies associated with the ontology traversal technique, and perform one or more actions associated with the clinical data based at least in part on the score for the relationship between the first medical concept and the second medical concept. The ontology traversal technique traverses an ontology knowledge graph of interlinked medical concepts with a hierarchy of class levels.

In accordance with yet another embodiment, a computer program product is provided. The computer program product can comprise at least one non-transitory computer-readable storage medium comprising instructions, the instructions being configured to cause one or more processors to at least perform operations configured to: extract one or more medical concepts from clinical data based at least in part on a natural language processing technique, identify corresponding clinical context for a first medical concept and a second medical concept in the clinical data by analyzing one or more medical ontologies based at least in part on an ontology traversal technique associated with a neural network, generate a score for a relationship between the first medical concept and the second medical concept based at least in part on one or more rules with respect to coded entries of the clinical data and the one or more medical ontologies associated with the ontology traversal technique, and perform one or more actions associated with the clinical data based at least in part on the score for the relationship between the first medical concept and the second medical concept. The ontology traversal technique traverses an ontology knowledge graph of interlinked medical concepts with a hierarchy of class levels.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of an architecture that can be used to practice one or more embodiments of the present invention.

FIG. 2 provides an example artificial intelligence computing entity in accordance with one or more embodiments discussed herein.

FIG. 3 provides an example external computing entity in accordance with one or more embodiments discussed herein.

FIG. 4 provides an example system associated with clinical data semantic interoperability using machine learning in accordance with one or more embodiments discussed herein.

FIG. 5 provides another example system associated with clinical data semantic interoperability using machine learning in accordance with one or more embodiments discussed herein.

FIG. 6 provides an example system associated with an ontology traversal technique in accordance with one or more embodiments discussed herein.

FIG. 7 provides an example system associated with decisions performed by a decision management engine in accordance with one or more embodiments discussed herein.

FIGS. 8A and 8B provide an example system associated with clinical data semantic interoperability using machine learning in accordance with one or more embodiments discussed herein.

FIG. 9 is a flowchart diagram of an example process for facilitating clinical data semantic interoperability using machine learning in accordance with one or more embodiments discussed herein.

### DETAILED DESCRIPTION

Various embodiments of the present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout. Moreover, while certain embodiments of the present invention are described with reference to predictive data analysis, one of ordinary skill in the art will recognize that the disclosed concepts can be used to perform other types of data analysis.

### I. Overview

Discussed herein are methods, apparatus, systems, computing devices, computing entities, and/or the like to facilitate clinical data semantic interoperability using artificial intelligence. As will be recognized, the disclosed concepts can be used to perform any type of artificial intelligence for clinical data semantic interoperability. Examples of artificial intelligence include, but are not limited to, machine learning, supervised machine learning, unsupervised machine learning, deep learning, neural network architectures, and/or the like.

Healthcare organizations often employ disparate network systems (e.g., disparate health internet technology systems) to facilitate providing one or more services. With advancement of technology and adoption of healthcare standards, it is desirable to consolidate meaningful health information from the disparate network systems. However, it is generally difficult and/or inefficient to obtain meaningful health information from the disparate network systems.

Various embodiments of the present invention address technical challenges related to accurately, efficiently and/or reliably consolidating health information from disparate network systems. In an aspect, semantic interoperability can be employed to consolidate meaningful health information. In another aspect, a machine learning solution is provided to improve quality and/or usefulness of clinical documents through incremental semantic interoperability. In certain embodiments, a reference information library (RIM) can also be employed by the machine learning solution to quality and/or usefulness of clinical documents. In certain embodiments, clinical information included in narrative sections of clinical document architecture (CDA) documents (e.g., clinical notes, case notes, discharge summary, and/or the like) can be interpreted. Furthermore, clinical context of the clinical information can be derived and/or accuracy of the clinical information can be established. Additionally, decisions can be configured based at least in part on the level of accuracy to auto-ingest the clinical information and/or initiate further clinical review of the clinical information.

In an embodiment, medical concepts embedded in the clinical documents can be identified using one or more computer-based algorithms. The medical concepts can also be segmented into relevant medical ontologies. In another embodiment, the medical concepts can be contextualized and/or one or more medical ontologies can be derived using an ontology traversal technique. A rule-based inference engine can also be employed to establish clinical accuracy of terms and/or phrases associated with the medical concepts. Furthermore, a decision management system manages one or more decisions based at least in part on accuracy scores provided by the rule-based inference engine in order to invoke one or more actions and/or one or more workflows for the healthcare organization.

In certain embodiments, natural language processing (NLP), a Medical Dictionary (MD), a Medical Thesaurus (MT) and/or one or more statistical machine learning techniques can be employed to extract one or more medical concepts, one or more modifiers, one or more assertions and/or one or more relationships from structured data and/or unstructured data associated with the clinical documents. Additionally, learning can be performed to provide understanding with respect to healthcare concepts and/or to establish clinical context. In certain embodiments, normalization of structured data and/or unstructured data can be performed, identified concepts can be matched, and/or meaningful information can be derived by executing one or more cross-section specific rules. In an embodiment related to unmatched root concepts, normalization of unstructured data can be performed, interlinked concept weightage can be identified from population data (e.g., for interlinked concepts which are present in a clinical document), and/or respective concept-wise scores can be derived.

Accordingly, by employing various techniques for consolidating health information from disparate network systems, various embodiments of the present invention enable utilizing efficient and reliable machine learning solutions to process high-dimensional categorical feature spaces with a high degree of size, diversity and/or cardinality. In doing so, various embodiments of the present invention address shortcomings of existing system solutions and enable solutions that are capable of accurately, efficiently and/or reliably performing predictive data analysis related to optimal consolidation and/or analysis of health information from disparate network systems to facilitate optimal decisions and/or actions related to the health information. By employing various techniques for consolidating health information from disparate network systems, various embodiments of the present invention also enable semantic interoperability to organize narrative text for a computer to interpret and/or perform one or more operations. For instance, by employing various techniques for consolidating health information from disparate network systems, narrative blocks of a clinical document can be interpreted and/or related to coded entries to establish completeness and/or correctness of the clinical data. As such, occurrences of incomplete portions of data (e.g., due to improper coding, and/or the like) for clinical data can be reduced. Moreover, by employing various techniques for consolidating health information from disparate network systems, one or more other technical benefits can be provided, including improved interoperability, improved reasoning, reduced errors, improved information/data mining, improved analytics, and/or the like.

### II. Computer Program Products, Methods, and Computing Entities

Embodiments of the present invention may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations. Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### III. Exemplary System Architecture

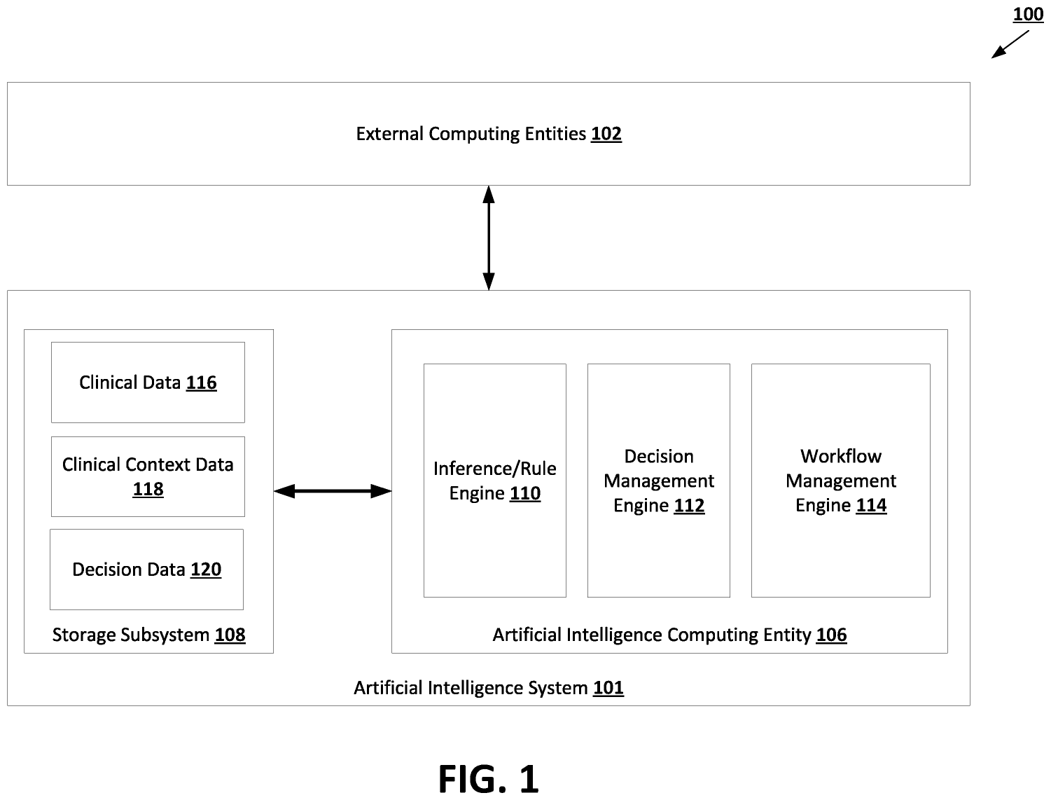

FIG. 1 provides an exemplary overview of an architecture 100 that can be used to practice embodiments of the present invention. The architecture 100 includes an artificial intelligence system 101 and one or more external computing entities 102. For example, at least some of the one or more external computing entities 102 can provide medical records inputs and/or clinical document inputs to the artificial intelligence system 101 and receive decision outputs, task outputs and/or action outputs from the artificial intelligence system 101 in response to providing the medical records inputs and/or clinical document inputs. As another example, at least some of the external computing entities 102 can provide one or more data streams and/or one or more batch loads to the artificial intelligence system 101 and request performance of particular prediction-based actions in accordance with the provided one or more data streams and/or one or more batch loads. As a further example, at least some of the external computing entities 102 can provide training data to the artificial intelligence system 101 and request training of a predictive model (e.g., a predictive machine learning model) in accordance with the provided training data. In some of the noted embodiments, the artificial intelligence system 101 can be configured to transmit parameters, hyper-parameters, and/or weights of a trained machine learning model to the external computing entities 102.

In some embodiments, the artificial intelligence system 101 can include an artificial intelligence computing entity 106. The artificial intelligence computing entity 106 and the external computing entities 102 can be configured to communicate over a communication network (not shown). The communication network can include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

Additionally, in some embodiments, the artificial intelligence system 101 can include a storage subsystem 108. The artificial intelligence computing entity 106 can be configured to provide one or more predictions using one or more artificial intelligence techniques. For instance, the artificial intelligence computing entity 106 can be configured to compute optimal decisions, display optimal data for a dashboard (e.g., a graphical user interface), generate optimal data for reports, optimize actions, and/or optimize configurations associated with a decision management system and/or a workflow management system. In some embodiments, an inference/rule engine 110 of the artificial intelligence computing entity 106 can determine one or more inferences associated with clinical data 116. The clinical data 116 can include structured data and/or unstructured data. In an embodiment, the clinical data 116 can include data from one or more clinical documents. For example, the clinical data 116 can include clinical information obtained from one or more narrative sections of one or more clinical documents (e.g., one or more CDA documents). The one or more narrative sections can include, for example, one or more clinical notes, one or more case notes, one or more discharge summaries and/or one or more other sections of one or more clinical documents. In another aspect, the clinical data 116 can be provided by disparate network systems (e.g., disparate health internet technology systems) associated with the one or more external computing entities 102.

In an aspect, the inference/rule engine 110 of the artificial intelligence computing entity 106 can employ one or more artificial intelligence techniques and/or one or more machine learning models to determine the one or more inferences associated with clinical data 116. Additionally, in some embodiments, the inference/rule engine 110 of the artificial intelligence computing entity 106 can employ model data that includes one or more machine learning models for consolidating and/or analyzing the clinical data 116. Furthermore, the inference/rule engine 110 of the artificial intelligence computing entity 106 can generate clinical context data 118 related to the one or more inferences associated with clinical data 116. The clinical context data 118 can provide, for example, semantic interoperability related to the clinical data 116. Furthermore, the clinical context data 118 can clinical context from multiple sections of the clinical data 116. For example, the clinical context data 118 can determine first clinical context for a first section of the clinical data 116 that is associated with a first medical concept, second clinical context for a second section of the clinical data 116 that is associated with a second medical concept, and/or the like. In an aspect, the clinical context data 118 can identify corresponding clinical context for a first medical concept and a second medical concept in the clinical data 116 by analyzing one or more medical ontologies based at least in part on an ontology traversal technique associated with a neural network. The ontology traversal technique can, for example, traverse an ontology knowledge graph of interlinked medical concepts with a hierarchy of class levels. In certain embodiments, the inference/rule engine 110 of the artificial intelligence computing entity 106 can identify one or more medical concepts embedded in the clinical data 116. The term “medical concept” and/or the like may refer to ideas associated with synonymous linguistic expressions of the medical concept. Each medical concept is described in direct or indirect relationship to the root node of a medical ontology. Furthermore, a medical concept description can reference another medical concept to serve as its anchor and then specify or redefine additional attributes (commonly referred to as “extra bits”) to distinguish it from the medical concept to which it is anchored. These attributes may specify membership in additional sets (some) or specify constraint to specific values (min, max, value). In one embodiment, a medical concept can be associated with a source vocabulary, a source vocabulary code, a description, and/or an identifier.

The inference/rule engine 110 of the artificial intelligence computing entity 106 can also segment the one or more medical concepts into one or more medical ontologies. A medical ontology is a representation, a terminology and/or a description that describes a portion of the clinical data 116. A medical ontology can additional describe a relationship between one or more other medical ontologies. In an embodiment, a medical ontology (in the context of computerized information systems) is a formal way of representing what things are or what they mean. A graph-based ontology can be used, for example, to represent concepts in a graph-based data structure. The concepts are then used to store information/data. In a graph-based medical ontology, description logic uses formal descriptions to represent concepts. These descriptions (e.g., class expressions) are constructed from less complex class expressions and relationships using formal operations such as AND, OR, SOME, and/or ALL. In an aspect, the inference/rule engine 110 of the artificial intelligence computing entity 106 can contextualize the one or more medical concepts embedded in the clinical data 116 to determine the one or more other medical ontologies.

In an embodiment, the inference/rule engine 110 of the artificial intelligence computing entity 106 can employ an ontology traversal technique to facilitate generation of the clinical context data 118. The ontology traversal technique can be a technique to traverse one or more portions of the clinical data 116 and/or an ontology knowledge graph of interlinked medical concepts with a hierarchy of class levels. For example, in an embodiment, the inference/rule engine 110 of the artificial intelligence computing entity 106 can search for one or more matching concepts within one or more groups and/or one or more hierarchies associated with the clinical data 116. In certain embodiments, the inference/rule engine 110 of the artificial intelligence computing entity 106 can traverse from a super-class level to a sub-class level associated with one or more groups and/or one or more hierarchies related to the clinical data 116. Additionally or alternatively, the inference/rule engine 110 of the artificial intelligence computing entity 106 can traverse from a sub-class level to a super-class level associated with one or more groups and/or one or more hierarchies related to the clinical data 116. As such, the inference/rule engine 110 of the artificial intelligence computing entity 106 can determine whether narrative text in the clinical data 116 matches one or more medical concepts. In an aspect, the ontology traversal technique can employ one or more ontology relations and/or one or more ontology rules to determine matches between narrative text in the clinical data 116 and one or more medical concepts. The term “traversal,” “traverse,” and/or the like may refer to a ontology knowledge graph. A traversal is a form of a graph traversal and indicates the process of visiting (checking, updating, executing methods, and/or the like) each node in the ontology knowledge graph. Such traversals are classified by the order in which the nodes are visited. Such traversals include depth-first searches/traversals (e.g., preorder, inorder, postorder,), breadth-first searches/traversals, and/or the like.

In another embodiment, the inference/rule engine 110 of the artificial intelligence computing entity 106 can employ a set of rules to facilitate validation of the ontology traversal. The set of rules can, for example, facilitate assessment of a level of accuracy of a match between narrative text in the clinical data 116 and one or more medical concepts associated with the ontology traversal. In certain embodiments, the inference/rule engine 110 of the artificial intelligence computing entity 106 can employ a set of weights and/or scoring defined for one or more health parameters and/or one or more health criteria. For instance, a score can indicate whether or not a portion of the clinical data 116 matches one or more health parameters and/or one or more health criteria. In certain implementations, a higher score can correspond to a greater degree of accuracy. In an embodiment, the inference/rule engine 110 of the artificial intelligence computing entity 106 can generate a score for a relationship between the first medical concept and the second medical concept included in the clinical context data 118 based at least in part on one or more rules with respect to coded entries of the clinical data 116 and the one or more medical ontologies associated with the ontology traversal technique.

In some embodiments, a decision management engine 112 of the artificial intelligence computing entity 106 can manage one or more decisions and/or one or more actions based at least in part on the clinical context data 118. The decision management engine 112 of the artificial intelligence computing entity 106 can, for example, generate decision data 120 that includes data related to the one or more decisions and/or one or more actions. In an embodiment, the decision management engine 112 of the artificial intelligence computing entity 106 can define one or more actions with respect to one or more matches between narrative text in the clinical data 116 and one or more medical concepts associated with the ontology traversal. In another embodiment, the decision management engine 112 of the artificial intelligence computing entity 106 can perform one or more actions associated with the clinical data 116 based at least in part on the score for the relationship between the first medical concept and the second medical concept included in the medical context data 118. In certain implementations, the decision management engine 112 of the artificial intelligence computing entity 106 can facilitate automated generation and/or storage of narrative text associated with the one or more decisions and/or one or more actions. Additionally or alternatively, the decision management engine 112 of the artificial intelligence computing entity 106 can facilitate rendering of data associated with the one or more decisions and/or one or more actions via a graphical user interface of a computing device. Additionally or alternatively, the decision management engine 112 of the artificial intelligence computing entity 106 can initiate one or more workflows based at least in part on the one or more decisions and/or one or more actions.

In some embodiments, a workflow management engine 112 of the artificial intelligence computing entity 106 can initiate one or more workflows based at least in part on the one or more decisions and/or one or more actions provided by the decision management engine 112. In an embodiment, the workflow management engine 112 of the artificial intelligence computing entity 106 can configure the one or more workflows to facilitate user review to validate matches between narrative text and ontologies. Additionally, in certain embodiments, the workflow management engine 112 of the artificial intelligence computing entity 106 can manage a graphical user interface of a computing device (e.g., a summary dashboard associated with a graphical user interface). For instance, the workflow management engine 112 of the artificial intelligence computing entity 106 can manage access to a graphical user interface of a computing device (e.g., a summary dashboard associated with a graphical user interface). Additionally or alternatively, the workflow management engine 112 of the artificial intelligence computing entity 106 can manage content rendered on a graphical user interface of a computing device (e.g., a summary dashboard associated with a graphical user interface).

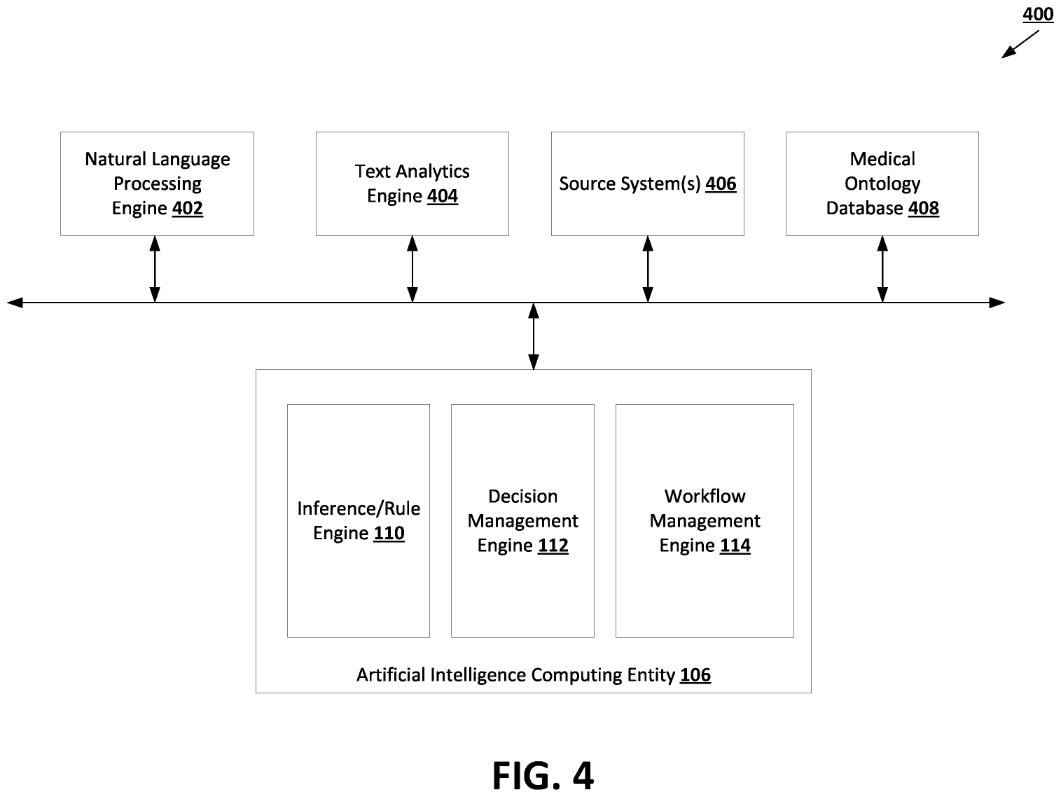

As such, the artificial intelligence computing entity 106 can provide accurate, efficient and/or reliable predictive data analysis for consolidating and/or analyzing the clinical data 116. Further example operations of the inference/rule engine 110, the decision management engine 112 and/or the workflow management engine 114 are described with reference to FIGS. 4-10. In an embodiment, the clinical data 116, the clinical context data 118 and/or the decision data 120 can be stored in a storage subsystem 108. The storage subsystem 108 can include one or more storage units, such as multiple distributed storage units that are connected through a computer network. Each storage unit in the storage subsystem 108 can store at least one of one or more data assets and/or one or more data about the computed properties of one or more data assets. Moreover, each storage unit in the storage subsystem 108 can include one or more non-volatile storage or memory media including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

Various embodiments provide technical solutions to technical problems corresponding to reasoning logic and reasoners for ontologies. In particular, the execution of reasoning logic tends to be resource intensive and time intensive. For example, continually querying a data structure would significantly slow down a data ingestion processes and/or would require significantly more computational resources. However, with the system 100 and one or more other embodiments disclosed herein, one or more technical improvements can be provided such as a reduction in computationally intensiveness and time intensiveness needed for automated managing, ingesting, monitoring, updating, and/or extracting/retrieving of clinical data (e.g., the clinical data 116). With the system 100 and one or more other embodiments disclosed herein, reduction in computational resources required for automated managing, ingesting, monitoring, updating, and/or extracting/retrieving of clinical data (e.g., the clinical data 116) can also be provided. The system 100 can also allocate processing resources, memory resources, and/or other computational resources to other tasks while executing one or more processes related to clinical data semantic interoperability in parallel. Improved clinical data can also be provided. As such, various embodiments of the present invention therefore provide improvements to the technical field of processing and/or analyzing health information from disparate network systems. In certain embodiments, a graphical user interface of a computing device that renders at least a portion of the clinical data can also be improved.

A. Exemplary Artificial Intelligence Computing Entity

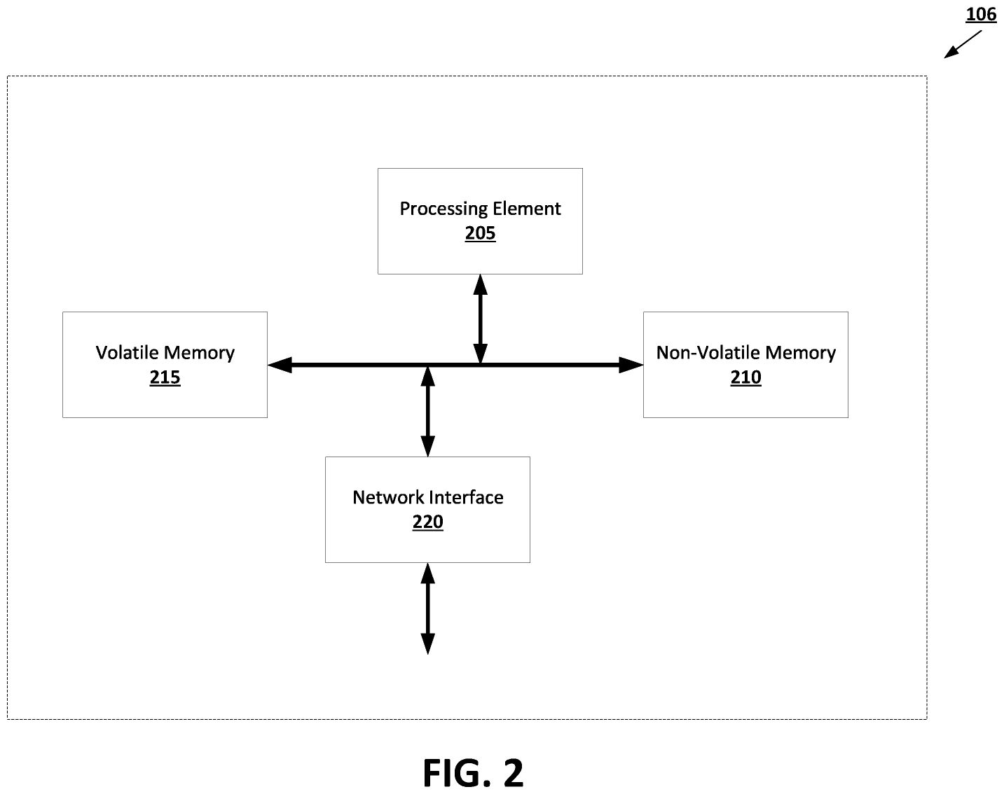

FIG. 2 provides a schematic of the artificial intelligence computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes can include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the artificial intelligence computing entity 106 can also include a network interface 220 for communicating with various computing entities, such as by communicating data, content, information, and/or similar terms used herein interchangeably that can be transmitted, received, operated on, processed, displayed, stored, and/or the like. Furthermore, it is to be appreciated that the network interface 220 can include one or more network interfaces.

As shown in FIG. 2, in one embodiment, the artificial intelligence computing entity 106 can include or be in communication with processing element 205 (also referred to as processors, processing circuitry, and/or similar terms used herein interchangeably) that communicate with other elements within the artificial intelligence computing entity 106 via a bus, for example. It is to be appreciated that the processing element 205 can include one or more processing elements. As will be understood, the processing element 205 can be embodied in a number of different ways. For example, the processing element 205 can be embodied as one or more complex programmable logic devices (CPLDs), microprocessors, multi-core processors, coprocessing entities, application-specific instruction-set processors (ASIPs), microcontrollers, and/or controllers. Further, the processing element 205 can be embodied as one or more other processing devices or circuitry. The term circuitry can refer to an entirely hardware embodiment or a combination of hardware and computer program products. Thus, the processing element 205 can be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 205 can be configured for a particular use or configured to execute instructions stored in volatile or non-volatile media or otherwise accessible to the processing element 205. As such, whether configured by hardware or computer program products, or by a combination thereof, the processing element 205 can be capable of performing steps or operations according to embodiments of the present invention when configured accordingly.

In one embodiment, the artificial intelligence computing entity 106 can further include or be in communication with non-volatile memory 210. The non-volatile memory 210 can be non-volatile media (also referred to as non-volatile storage, memory, memory storage, memory circuitry and/or similar terms used herein interchangeably). Furthermore, in an embodiment, non-volatile memory 210 can include one or more non-volatile storage or memory media, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. As will be recognized, the non-volatile storage or memory media can store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably can refer to a collection of records or data that is stored in a computer-readable storage medium using one or more database models, such as a hierarchical database model, network model, relational model, entity-relationship model, object model, document model, semantic model, graph model, and/or the like.

In one embodiment, the artificial intelligence computing entity 106 can further include or be in communication with volatile memory 215. The volatile memory 215 can be volatile media (also referred to as volatile storage, memory, memory storage, memory circuitry and/or similar terms used herein interchangeably). Furthermore, in an embodiment, the volatile memory 215 can include one or more volatile storage or memory media, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. As will be recognized, the volatile storage or memory media can be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like can be used to control certain aspects of the operation of the artificial intelligence computing entity 106 with the assistance of the processing element 205 and operating system.

As indicated, in one embodiment, the artificial intelligence computing entity 106 can also include the network interface 220. In an embodiment, the network interface 220 can be one or more communications interfaces for communicating with various computing entities, such as by communicating data, content, information, and/or similar terms used herein interchangeably that can be transmitted, received, operated on, processed, displayed, stored, and/or the like. Such communication can be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the artificial intelligence computing entity 106 can be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the artificial intelligence computing entity 106 can include or be in communication with one or more input elements, such as a keyboard input, a mouse input, a touch screen/display input, motion input, movement input, audio input, pointing device input, joystick input, keypad input, and/or the like. The artificial intelligence computing entity 106 can also include or be in communication with one or more output elements (not shown), such as audio output, video output, screen/display output, motion output, movement output, and/or the like.

B. Exemplary External Computing Entity

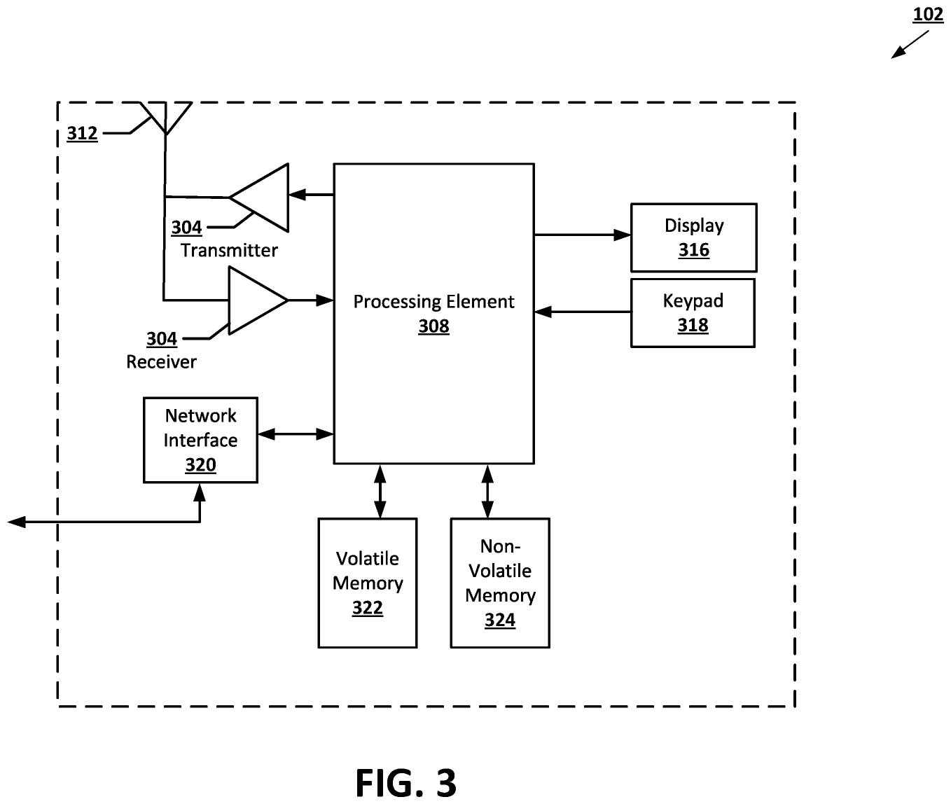

FIG. 3 provides an illustrative schematic representative of an external computing entity 102 that can be used in conjunction with embodiments of the present invention. In general, the terms device, system, computing entity, entity, and/or similar words used herein interchangeably can refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. The external computing entity 102 can be operated by various parties. As shown in FIG. 3, the external computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, can include signaling information/data in accordance with air interface standards of applicable wireless systems. In this regard, the external computing entity 102 can be capable of operating with one or more air interface standards, communication protocols, modulation types, and access types. More particularly, the external computing entity 102 can operate in accordance with any of a number of wireless communication standards and protocols, such as those described above with regard to the artificial intelligence computing entity 106. In a particular embodiment, the external computing entity 102 can operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the external computing entity 102 can operate in accordance with multiple wired communication standards and protocols, such as those described above with regard to the artificial intelligence computing entity 106 via a network interface 320.

Via these communication standards and protocols, the external computing entity 102 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The external computing entity 102 can also download changes, add-ons, and updates, for instance, to its firmware, software (e.g., including executable instructions, applications, program modules), and operating system.

According to one embodiment, the external computing entity 102 can include location determining aspects, devices, modules, functionalities, and/or similar words used herein interchangeably. For example, the external computing entity 102 can include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites can be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the external computing entity's 102 position in connection with a variety of other systems, including cellular towers, Wi-Fi access points, and/or the like. Similarly, the external computing entity 102 can include indoor positioning aspects, such as a location module adapted to acquire, for example, latitude, longitude, altitude, geocode, course, direction, heading, speed, time, date, and/or various other information/data. Some of the indoor systems can use various position or location technologies including RFID tags, indoor beacons or transmitters, Wi-Fi access points, cellular towers, nearby computing devices (e.g., smartphones, laptops) and/or the like. For instance, such technologies can include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The external computing entity 102 can also comprise a user interface (that can include a display 316 coupled to the processing element 308) and/or a user input interface (coupled to the processing element 308). For example, the user interface can be a user application, browser, user interface, graphical user interface, dashboard, and/or similar words used herein interchangeably executing on and/or accessible via the external computing entity 102 to interact with and/or cause display of information/data from the artificial intelligence computing entity 106, as described herein. The user input interface can comprise any of a number of devices or interfaces allowing the external computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the external computing entity 102 and can include a full set of alphabetic keys or set of keys that can be activated to provide a full set of alphanumeric keys. In addition to providing input, the user input interface can be used, for example, to activate or deactivate certain functions, such as screen savers and/or sleep modes.

The external computing entity 102 can also include volatile memory 322 and/or non-volatile memory 324, which can be embedded and/or can be removable. For example, the non-volatile memory can be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory can be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile memory 322 and/or the non-volatile memory 324 can store databases, database instances, database management systems, data, applications, programs, program modules, scripts, source code, object code, byte code, compiled code, interpreted code, machine code, executable instructions, and/or the like to implement the functions of the external computing entity 102. As indicated, this can include a user application that is resident on the entity or accessible through a browser or other user interface for communicating with the artificial intelligence computing entity 106 and/or various other computing entities.

In another embodiment, the external computing entity 102 can include one or more components or functionality that are the same or similar to those of the artificial intelligence computing entity 106, as described in greater detail above. As will be recognized, these architectures and descriptions are provided for exemplary purposes only and are not limiting to the various embodiments.

In various embodiments, the external computing entity 102 can be embodied as an artificial intelligence (AI) computing entity, such as a virtual assistant AI device, and/or the like. Accordingly, the external computing entity 102 can be configured to provide and/or receive information/data from a user via an input/output mechanism, such as a display, a camera, a speaker, a voice-activated input, and/or the like. In certain embodiments, an AI computing entity can comprise one or more predefined and executable program algorithms stored within an onboard memory storage module, and/or accessible over a network. In various embodiments, the AI computing entity can be configured to retrieve and/or execute one or more of the predefined program algorithms upon the occurrence of a predefined trigger event.

### IV. Exemplary System Operations

In general, embodiments of the present invention provide methods, apparatus, systems, computing devices, computing entities, and/or the like for recommendation prediction using artificial intelligence. Certain embodiments of the systems, methods, and computer program products that facilitate recommendation prediction employ one or more machine learning models and/or one or more machine learning techniques.

Various embodiments of the present invention address technical challenges related to accurately, efficiently and/or reliably performing predictive data analysis in prediction domains. For example, in some embodiments, proposed solutions disclose a recommendation system to match an entity with an optimal selection of ranked results to satisfy criteria for one or more goals for the entity. In some embodiments, proposed solutions disclose an optimal recommendation of a provider entity from a set of provider entities. In some embodiments, a machine learning model to facilitate the optimal recommendation can be generated based at least in part on embeddings for respective provider entities. After the machine learning model is generated, the machine learning model can be utilized to perform accurate, efficient and reliable predictive data analysis related to optimal recommendation of a provider entity from a set of provider entities.

### Clinical Data Semantic Interoperability Using Machine Learning

FIG. 4 illustrates an example system 400 for clinical data semantic interoperability using machine learning. In an embodiment, the system 400 can provide for consolidating and/or analyzing clinical data using machine learning, natural language processing and/or text analytics. The system 400 includes the artificial intelligence computing entity 106. The artificial intelligence computing entity 106 includes the inference/rule engine 110, the decision management engine 112 and/or the workflow management engine 114. The system 400 additionally includes a natural language processing engine 402, one or more source systems 406 and/or a medical ontology database 408.

In an embodiment, the one or more source systems 406 can be related to disparate network systems (e.g., disparate health internet technology systems). For instance, in an embodiment, the one or more source systems 406 can include one or more database systems, one or more server systems, one or more electronic record data store systems, one or more claims processing systems, one or more laboratory computing entities, one or more user computing entities, and/or one or more other source systems. Furthermore, the one or more source systems 406 can provide at least a portion of the clinical data 116. In an embodiment, the one or more source systems 406 can be associated with the one or more external computing entities 102. The clinical data 116 can be stored in the one or more source systems 406 as structured data and/or unstructured data, for example. In another embodiment, the natural language processing engine 402 can perform one or more natural language processing techniques to extract textual data from the clinical data 116 associated with the one or more source systems 406. In an embodiment, the natural language processing engine 402 can perform one or more text recognition techniques to extract textual data from the clinical data 116 associated with the one or more source systems 406. In an example, the natural language processing engine 402 can perform one or more text recognition techniques to identify one or more words, one or more phrases, and/or one or more identifiers included in the clinical data 116. The one or more natural language processing techniques employed by the natural language processing engine 402 can include one or more machine learning techniques and/or one or more deep learning techniques. For instance, the natural language processing engine 402 can process natural language information/data (e.g., human language data, such as unstructured human language data) associated with the clinical data 116 to extract one or more features from the natural language information/data and/or to perform one or more predictions based at least in part on the natural language information/data. The natural language processing engine 402 can also employ one or more syntactic processing techniques and/or one or more semantic processing techniques. For example, natural language processing engine 402 can employ one or more of the following techniques: grammar induction, lemmatization, morphological segmentation, part-of-speech tagging, parsing, sentence boundary disambiguation, word segmentation, terminology extraction, lexical semantic determination, named entity recognition, optical character recognition, textual entailment recognition, relationship extraction, sentiment analysis, topic segmentation, word sense disambiguation, automatic segmentation, speech segmentation, text-to-speech conversion, and/or the like. In an embodiment, the natural language processing engine 402 can extract one or more medical concepts, one or more modifiers and/or other medical information from the clinical data 116 using the one or more natural language processing techniques. In another embodiment, the natural language processing engine 402 can employ a medical dictionary to facilitate extraction of one or more medical concepts, one or more modifiers and/or other medical information from the clinical data 116. For instance, the natural language processing engine 402 can compare textual data of the clinical data 116 with one or more terms included in the medical dictionary. In another example, the natural language processing engine 402 can expand one or more abbreviations included in the textual data of the clinical data 116 based at least in part on one or more terms included in the medical dictionary.

The natural language processing engine 402 can additionally perform one or more text analytics techniques to interpret textual data identified by the natural language processing engine 402. For instance, the natural language processing engine 402 can derive meaning from textual data associated with the clinical data 116. The natural language processing engine 402 can employ one or more machine learning techniques and/or one or more deep learning techniques to derive meaning from the textual data identified by the natural language processing engine 402. For instance, the natural language processing engine 402 can employ one or more natural language understanding techniques, one or more natural language generation techniques, and/or one or more statistical machine learning techniques to derive meaning from the textual data identified by the natural language processing engine 402. In an embodiment, the natural language processing engine 402 can extract one or more assertions, one or more relationships and/or other inferences associated with the textual data identified by the natural language processing engine 402 using the one or more text analytics techniques. In another embodiment, the natural language processing engine 402 can employ a medical thesaurus to interpret textual data identified by the natural language processing engine 402. For instance, the natural language processing engine 402 can employ the medical thesaurus to establish clinical context of the textual data identified by the natural language processing engine 402.

In a non-limiting example, a portion of the clinical data 116 (e.g., a narrative extract) can include textual data such as, for example, “She has been unable to tolerate multiple types of long acting pain medications, and also is intolerant of NSAIDs and COX-2 inhibitors. For now, she will continue Medication_1 and Medication_2, as she feels that these have worked the best for her in the past and other options are not available.” The natural language processing engine 402 can determine, for example, that “she” refers to a patient. The natural language processing engine 402 can also identify the phrase “unable to tolerate multiple types of long acting pain medications” and determine that “unable to tolerate” is related to “Type of Allergy.” The natural language processing engine 402 can also employ the medical thesaurus to identify one or more synonyms associated with “unable to tolerate.” Furthermore, the natural language processing engine 402 can also identify the phrase “intolerant of NSAIDs,” the phrase “intolerant of COX-2 inhibitors,” the phrase “continue Medication_1,” and the phrase “continue Medication_2.” The natural language processing engine 402 can also determine that Medication_1 is a substance, that one or more short acting pain medications is related to Medication_1, and that Medication_2 contains one or more other medications which are long action pain medications.

The inference/rule engine 110 can employ data from the natural language processing engine 402. In certain embodiments, the inference/rule engine 110 can generate the clinical context data 118 based at least in part on data from the natural language processing engine 402. Additionally, the inference/rule engine 110 can employ data from the natural language processing engine 402 to facilitate the ontology traversal technique. For instance, the inference/rule engine 110 can traverse the medical ontology database 408 based at least in part on data from the natural language processing engine 402. In an aspect, the inference/rule engine 110 can perform the ontology traversal technique to identify one or more root concepts and/or one or more corresponding interlinked concepts for one or more medical concepts identified by the natural language processing engine 402. In another aspect, a list of interlinked concepts can be employed to determine at least a portion of the clinical context data 118 by determining one or more related clinical sections.

Further to the non-limiting example described in connection with the natural language processing engine 402, a medical concept determined by the natural language processing engine 402 can be “long acting pain medication,” one or more modifiers determined by the natural language processing engine 402 can be “Long Acting” and “Multiple Types,” an assertion determined by the natural language processing engine 402 can be “Conditional (Patient is intolerant to long acting pain medications, Intolerance to Medication [CONDITION_1234]),” and a relation determined by the natural language processing engine 402 can be “Patient.” Furthermore, the ontology traversal technique performed by the inference/rule engine 110 can determine that the medical concept “long action pain medication” is not included in the medical ontology database 408 as a medical concept. However, the inference/rule engine 110 can determine that a “Pain Medication [MEDICATION_5678]” is included in the medical ontology database 408 and corresponds to the medical concept “long action pain medication.” As such, the inference/rule engine 110 can modify the medical concept “Pain Medication [MEDICATION_5678]” such that an aliasTerm=“long acting pain medication” is added to the medical concept “Pain Medication [MEDICATION_5678]” included in the medical ontology database 408.

Additionally, the ontology traversal technique performed by the inference/rule engine 110 can determine an optimal match for the medical concept “long action pain medication” based at least in part on the description “long action pain medication” and the description “Medication.” In an aspect, the medical concept “Pain Medication [MEDICATION_5678]” can be a root concept associated with the ontology traversal technique. The ontology traversal technique performed by the inference/rule engine 110 can also obtain a list of pain medications from a product form provided by the one or more source system 406 and/or a substance ontology included in the medical ontology database 408. In addition, the ontology traversal technique performed by the inference/rule engine 110 can identify one or more interlinked concepts from an ontology knowledge graph managed by the medical ontology database 408. The ontology knowledge graph can be a network of interlinked medical concepts with a hierarchy of class levels and/or interconnections that represent relationships between the medical concepts. In an embodiment, the ontology knowledge graph can be a non-linear data structure (e.g., a tree data structure, a container tree data structure, a container tree, and/or the like) where data objects are organized in terms of hierarchical relationships with a root value and subtrees of children with a parent node, represented as a set of linked nodes. For example, in the ontology knowledge graph, a node is a data object that may contain a value, condition, method for execution, reference, or represent a separate data structure or data object. The top node in the ontology knowledge graph may be referred to as the “root node.” A “child node” may be a node directly connected to another node when moving away from the root node, an immediate descendant. Similarly, a “parent node” may be the converse a child node, an immediate ancestor. A “leaf node” and/or the like may refer to a node with no children. An “internal node” and/or the like may refer to a node with at least one child. And “edge” and/or the like may refer to a connection between one node and another node. The “depth” of a node may refer to the distance between the particular node and the root node.

In another aspect, the ontology traversal technique performed by the inference/rule engine 110 can locate a parent concept from the ontology knowledge graph and/or can cache a list of child concepts associated with the parent concept. For instance, a parent concept from the ontology knowledge graph can be, for example, “Pain [CONDITION_45])” with a type that corresponds to a “Condition.” The list of child concepts can therefore be a list of conditions related to the parent concept “Pain.” Furthermore, the ontology traversal technique performed by the inference/rule engine 110 can additionally locate a parent concept for one or more other interlinked concepts. For example, the ontology traversal technique performed by the inference/rule engine 110 can locate another parent concept that correspond to “Drug Allergy [CONDITION 330],” and/or the like. The ontology traversal technique performed by the inference/rule engine 110 can additionally determine one or more relationships, such as a relationship between a “Drug Allergy” section of the clinical data 116 and a “Medication” of the clinical data 116, where “Pain Medication” is a drug and is related to Drug Allergy [CONDITION_330].” In an example, the ontology traversal technique performed by the inference/rule engine 110 can determine that Drug [SUBSTANCE_216]->has assertion->Intolerance [CONDITION_86]->Allergy/Adverse Reactions [OBSERVATION_58]. In another example, the ontology traversal technique performed by the inference/rule engine 110 can determine that Drug [SUBSTANCE_216]->has assertion->Intolerance [CONDITION_86]->Medication [OBSERVATION_544]. The terms “relationship” and/or the like can be a named, directed (one way) association between two concepts. A relationship establishes a semantic link between the respective concepts. Any given concept may function as both the source and destination of numerous relationships by establishing various named relationships. Thus, it is possible to have a variety of different structures-such as hierarchies, networks, and unstructured groups, superimposed on the same concepts. The ontology (represented as a directed acyclic graph data structure, a graph-based data structure, and/or the like) has the ability to be dynamically updated to modify relationships.

In another embodiment, the inference/rule engine 110 can match identified concepts associated with the medical ontology database 408 and/or can derive meaning associated with the identified concepts by executing one or more cross section specific rules. For example, the inference/rule engine 110 can match one or more root concepts to medical concepts. If a match is identified, the inference/rule engine 110 can also validate the information. In response to a determination that a root concept does not match a medical concept, the inference/rule engine 110 can analyze an information health record provided by the one or more source systems 406. The inference/rule engine 110 can, for example, match one or more root concepts to interlinked concepts in the information health record. If a match is identified, the inference/rule engine 110 can also validate the information in the information health record. Additionally, in certain embodiments, the inference/rule engine 110 can compare one or more modifiers, one or more assertions and/or one or more relationships to patient data provided by the one or more source systems 406. In response to a determination that a modifier, an assertion and/or a relationship does not correspond to data included in the patient data, a corresponding portion of the clinical data 116 can be provided to a graphical user interface to facilitate user review.

Further to the non-limiting example described in connection with the “Drug Allergy” section and “Medication” section determined by the inference/rule engine 110, the inference/rule engine 110 can match one or more root concepts to structured data associated with a clinical document. The inference/rule engine 110 can employ a list of concepts from a product form and substance ontology to match any concept associated with the “Drug Allergy” section and “Medication” section. The inference/rule engine 110 can also execute a specific parameter matching based at least in part on a type of section. In an example, the inference/rule engine 110 can determine that “Pain Medication Derivatives [SUBSTANCE_92]” is included the “Drug Allergy” section based at least in part on a concept “List of Pain Medications/Substance” and a type “Medications.” The inference/rule engine 110 can also employ one or more inferences such as, for example, “pain medication is absent in the list of allergens in the Drug Allergy Section,” “an independent class for long acting opioid is missing in the ontology,” and/or the like. In an example, the inference/rule engine 110 can determine that “Medication_1” is included in the “Medication” section but is classified as a “short acting” medication. Furthermore, the inference/rule engine 110 can determine that “Medication_2” is included in the “Medication” section but is classified as a “long acting” medication. The inference/rule engine 110 can also employ one or more inferences such as, for example, “Medication_1 and Medication_2 are a certain type of pain medication found in an active list of medications.”

An example of a cross section inference analysis performed by the inference/rule engine 110 can include cross section inference analysis between the “Drug Allergy” section and the “Medication” section. For example, the cross section inference analysis performed by the inference/rule engine 110 can determine that a patient associated with the clinical data 116 is allergic to pain medications and specifically to multiple types of long acting pain medications. The inference/rule engine 100 can additionally validate the cross section inference analysis in both the “Drug Allergy” section and the “Medication” section. Furthermore, the inference/rule engine 100 can correlate the information to derive a final inference. In an example, the inference/rule engine 110 can employ an inference “long acting pain medication should be present in the Drug Allergy section” with a rule “check if pain medication with a modifier long acting is present in the allergens list.” In another example, the inference/rule engine 110 can employ an inference “long acting pain medication should contain the reaction and the severity in allergy section” with a rule “check if the medication intolerance has the reaction and indicator severity parameter defined.” In yet another example, the inference/rule engine 110 can employ an inference “long acting pain medication should be present in the ontology as an independent class and therefore needs to create a new concept” with a rule “check if long acting pain medication (concept+modifier) is present as a child concept in the Pain Medication [MEDICATION_5678].” In yet another example, the inference/rule engine 110 can employ an inference “Medication_2 is a long acting pain medication and send corresponding information to the decision management engine 112 for clinical review” with a rule “check if patient active medication list contains any medication marked as allergic” and a recommendation “if long acting pain medication is present in the allergens list then add Medication_2 to the allergy list with a negation indicator.” In yet another example, the inference/rule engine 110 can employ an inference “Medication_1 is a short acting pain medication and do not send corresponding information to the decision management engine 112 for clinical review” with a rule “check if patient active medication list contains any medication marked as allergic” and a rule “check if Medication_1 is long acting (modifier).”

In another embodiment, for one or more unmatched root concepts, the inference/rule engine 110 can perform normalization of unstructured data. The inference/rule engine 110 can additionally or alternatively identify interlinked concept weightage from population data for interlinked concepts and/or features present in the clinical document. In certain embodiments, the inference/rule engine 110 can additionally or alternatively derive one or more concept-wise scores. The population data can be based at least in part on a number of patients associated with the one or more unmatched root concepts. An individual feature score can be based at least in part on a scale (e.g., from 0 to 10) that can be computed for each interlinked concept found in the clinical document using the population data. In certain embodiments, the inference/rule engine 110 can associate a strength of binding to root concepts based at least in part on one or more scores and/or a range of scores. For example, the inference/rule engine 110 can associate a root concept with a first score (e.g., STRONG), a second score (e.g., MODERATE) or a third score (e.g., WEAK). A score that corresponds to “STRONG” can indicate that the root concept has a strong association with one or more interlinked concepts based at least in part on population data (e.g., an allergy for a patient corresponds to having a certain type of medication as an allergen). Furthermore, a score (e.g., a strength of association) can be dynamically changed based at least in part on a change in the population data (e.g., a change in population data attribution).

Further to the non-limiting example described in connection with the “Drug Allergy” section determined by the inference/rule engine 110, the inference/rule engine 110 can determine certain medication derivatives in the “Drug Allergy” section from the concept “List of Medications/Substance) and the type “Medications.” In an example, the inference/rule engine 110 can employ one or more inferences such as, for example, “Medication_3 [MEDICATION_44] belongs to the pain medication group of medications,” “patients who are allergic to Medication_3 are potentially allergic to one or more pain medications from population data,” “a score 9 (90%) corresponds to a strong association,” “infer a very weak association between allergy to pain medications and patients on active medications Medication_1 and Medication_2,” “add long acting pain medications to the drug allergy section,” “recommend adding Medication_2 to the allergy list with a negation indicator,” and/or the like.

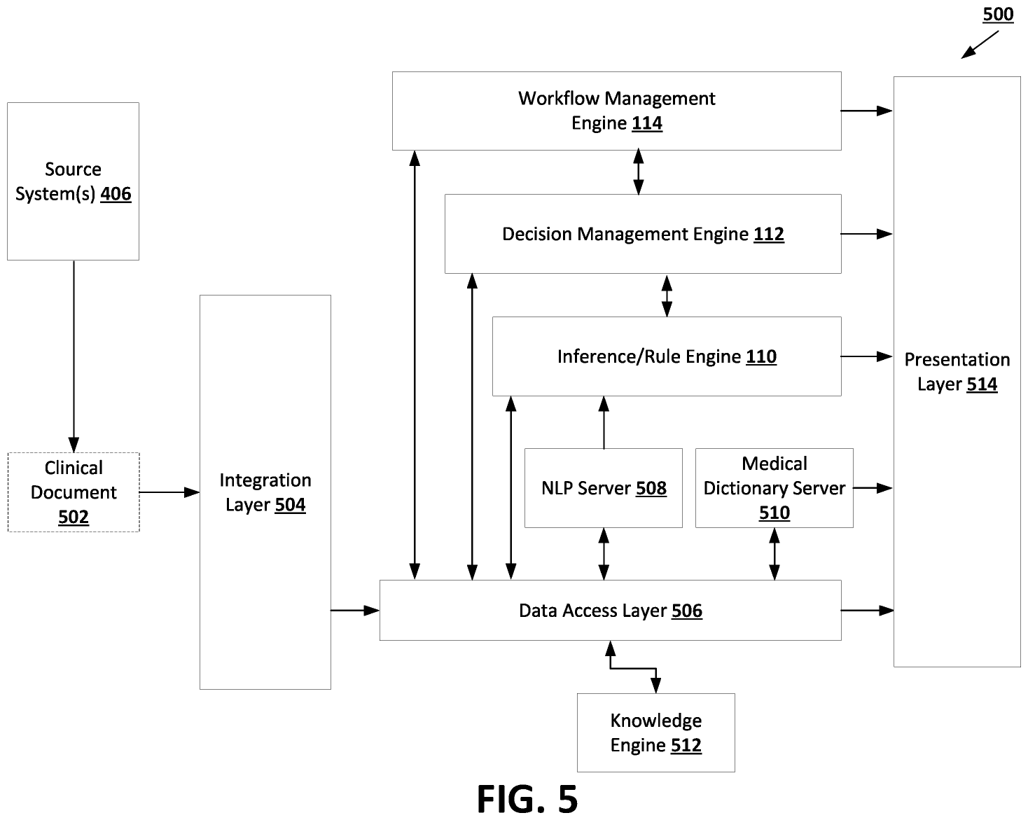

FIG. 5 illustrates an example system 500 for clinical data semantic interoperability using machine learning. In an embodiment, the system 500 can provide for consolidating and/or analyzing clinical data using machine learning, natural language processing and/or text analytics. The system 500 includes one or more components of the artificial intelligence computing entity 106. As shown in FIG. 5, the system 500 includes the inference/rule engine 110, the decision management engine 112 and the workflow management engine 114. In an embodiment, the one or more source systems 406 provide a clinical document 502. For instance, the clinical document 502 can correspond to one or more portions of the clinical data 116. In an aspect, clinical document 502 can include clinical information associated with one or more narrative sections of the clinical document 502. In one example, the clinical document 502 can be a CDA document. The one or more narrative sections of the clinical document 502 can include, for example, one or more clinical notes, one or more case notes, one or more discharge summaries and/or one or more other sections of the clinical document 502. In another aspect, the one or more source systems 406 can include one or more electronic medical record systems, one or more electronic health record systems, one or more healthcare systems, one or more laboratory information systems, one or more radiology information systems, one or more research information systems, one or more healthcare discharge systems.

The system 500 also includes an integration layer 504 that stores the clinical document 502 in a file repository, for example. The integration layer 504 can include one or more individual health record components, one or more ontology services, one or more interfaces, one or more core applications, one or more identity management systems, one or more integrated medical systems, one or more semantic single best record systems, one or more access management systems, and/or one or more other components. In certain embodiments, the integration layer 504 can store header information of the clinical document 502 in an application database. In an embodiment, the one or more identity management systems of the integration layer 504 can match and/or validate one or more patient demographics, enrollment data and/or eligibility data for a patient identity associated with the clinical document 502. In another embodiment, the one or more ontology services of the integration layer 504 can provide one or more relations of medical concepts. The one or more ontology services of the integration layer 504 can additionally or alternatively provide data associated with synonyms and/or aliases for one or more medical concepts. In yet another embodiment, the one or more semantic single best record systems can identify one or more medical records related to a patient identity associated with the clinical document 502. In an aspect, the one or more semantic single best record systems can include a key-value pair (e.g., ontology concept identifier and its associated/corresponding value) and/or corresponding metadata.

Additionally, the system 500 also includes a data access layer 506, an NLP server 508, a medical dictionary server 510, a knowledge engine 512 and/or a presentation layer 514. The data access layer 506 can manage data provided to and/or from the file repository, the application database, the NLP server 508, the medical dictionary server 510, the knowledge engine 512, the inference/rule engine 110, the decision management engine 112 and/or the workflow management engine 114. In an embodiment, the NLP server 508 can process the clinical document 502 using one or more natural language processing techniques to identify one or more medical concepts in the clinical document 502. The NLP server 508 can provide one or more computing resources for the natural language processing engine 402 and/or can include the natural language processing engine 402. In an aspect, the NLP server 508 can employ a medical dictionary and/or a medical thesaurus managed by the medical dictionary server 510 to identify the one or more medical concepts in the clinical document 502. In certain embodiments, the NLP sever 508 can additionally employ one or more assertion modifiers to identify the one or more medical concepts in the clinical document 502.

The inference/rule engine 110 can identify one or more medical ontology concepts and/or one or more related vocabularies in coded entries of the clinical document 502. Additionally or alternatively, the inference/rule engine 110 can identify one or more medical ontology concepts and/or one or more related vocabularies based at least in part on one or more medical ontology interlinked vocabularies associated with the knowledge engine 512. Furthermore, the inference/rule engine 110 can correlate one or more medical concepts to one or more probable candidates using the knowledge engine 512 and/or one or more related rules. The knowledge engine 512 can be, for example, a repository of clinical terms and/or associated metadata. Data of the knowledge engine 512 can be dynamic and/or updated in response to one or more changes and/or learning associated with the system 500. The decision management engine 112 can determine one or more decisions based at least in part on a set of rules and/or one or more defined thresholds. The presentation layer 514 can render data associated with the one or more decisions via a graphical user interface (e.g., a dashboard). The workflow management engine 114 can initiate one or more workflows based at least in part on the one or more decisions and/or one or more system configurations associated with the decision management engine 112. In an aspect, the presentation layer 514 can be accessed by one or more computing devices associated with clinical analysts, healthcare providers, system administrators and/or other users to view the graphical user interface (e.g., a dashboard), to generate one or more reports, to perform one or more actions and/or to perform one or more configurations.

In an embodiment, the inference/rule engine 110 includes an ontology traversal component and a validator component. The ontology traversal component of the inference/rule engine 110 can search for one or more matching medical concepts within a medical ontology. The ontology traversal component of the inference/rule engine 110 can also perform an ontology traversal technique that traverses from a super class to sub class level (and vice-versa) to identify one or more related concepts and/or to determine whether particular narrative text of the clinical document 502 matches one or more related concepts. In an aspect, the ontology traversal component of the inference/rule engine 110 can employ one or more ontology relations and/or one or more rules to determine an appropriate match. The validator component of the inference/rule engine 110 can employ a set of rules that are defined to assess a level of accuracy of matches between the narrative terms and the ontology traversal. Weightage and/or scoring can also be defined for one or more health parameters and/or health criteria. A score can be added in response to a determination that patient data associated with the clinical document 502 matches one or more of the defined parameters. A greater score can correspond to greater accuracy. In another embodiment, the decision management engine 112 can define one or more actions with respect to each of the matches based at least in part on the scores determined by the validator component of the inference/rule engine 110. Furthermore, an action can initiate one or more workflows associated with the workflow management engine 114. In yet another embodiment, the workflow management engine 114 can configure one or more workflows to validate text to ontology matches. The workflow management engine 114 can also allow one or more computing devices to access the graphical user interface associated with the presentation layer 514 based at least in part on one or more access roles. Providers and/or reviewers associated with the one or more computing devices can validate or make changes to the matches via the presentation layer 514 before finalizing the decisions.

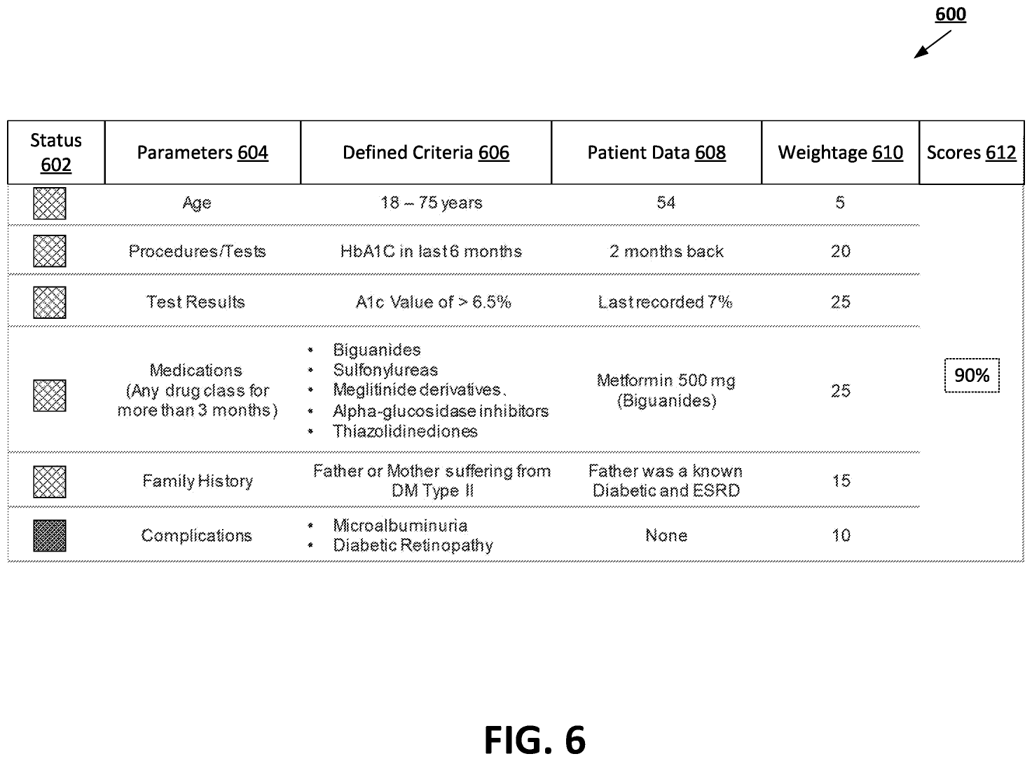

In some embodiments, the ontology traversal technique performed by the inference/rule engine 110 can be performed in accordance with a system 600 depicted in FIG. 6. For example, the system 600 can include example text data included in clinical data (e.g., the clinical data 116) and/or a clinical document (e.g., the clinical document 502). The text data can include a status 602, parameters 604, defined criteria 606, patient data 608, weightage 610, and/or scores 612. In certain embodiments, the status 602, the parameters 604, the defined criteria 606, the patient data 608, the weightage 610, and/or the scores 612 can be determined by the inference/rule engine 110. In an embodiment, the inference/rules engine 612 can determine data for the status 602, the parameters 604, the defined criteria 606, the patient data 608, the weightage 610, and/or the scores 612 to determine one or more parameters and/or one or more criteria that can establish a certain type of medical condition. In certain embodiments, the inference/rules engine 612 can assign a score for each parameter and/or can determine a cumulative accuracy score. In certain embodiments, the decision management engine 112 can manage one or more decisions based at least in part on the scores. In one example, a parameter “age” can include defined criterion “18-75 years” with patient data equal to “54” and weightage equal to “5%.”

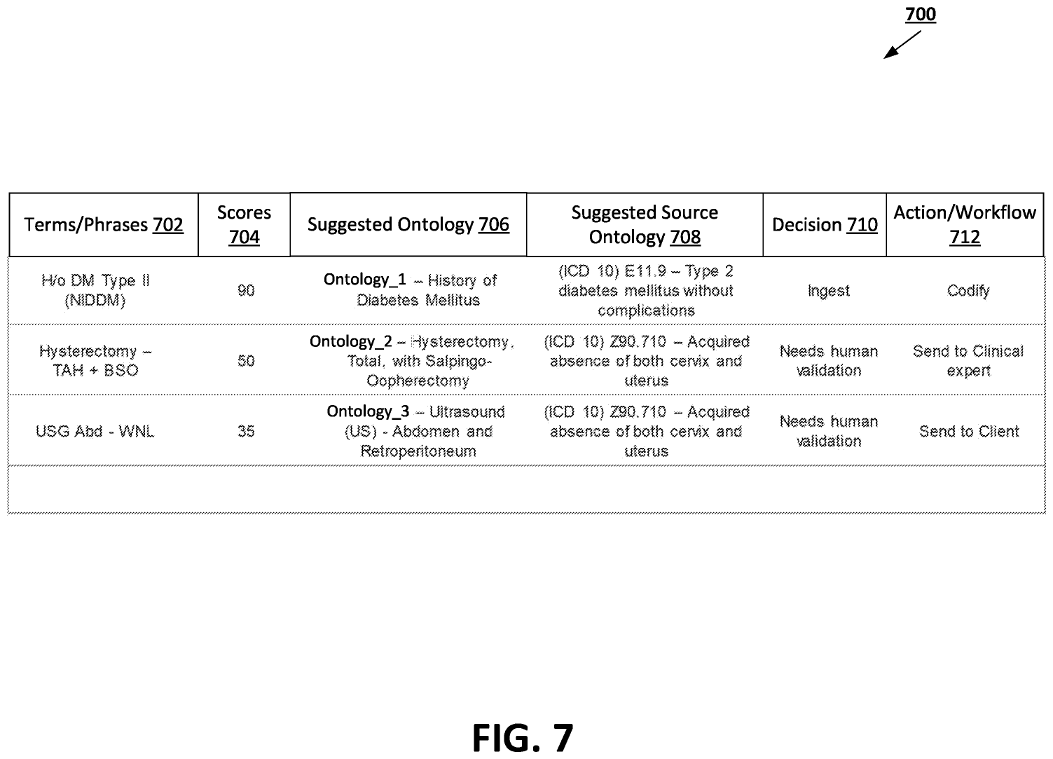

In some embodiments, one or more decisions can be performed by the decision management engine 112 in accordance with a system 700 depicted in FIG. 7. In an embodiment, the inference/rule engine 110 can determine terms/phrases 702, scores 704, a suggested ontology 706 and/or a suggested source ontology 708. Furthermore, the decision management engine 112 can determine a decision 710 and/or an action/workflow 712. In one example, a term/phrase “H/o DM Type II (NIDDM)” can include a score “90,” a suggest ontology “CDN 123—History of Diabetes Mellitus,” a suggested source ontology “(ICD 10) E11.9—Type 2 diabetes mellitus without complications,” a decision “ingest” and an action/workflow “codify.”

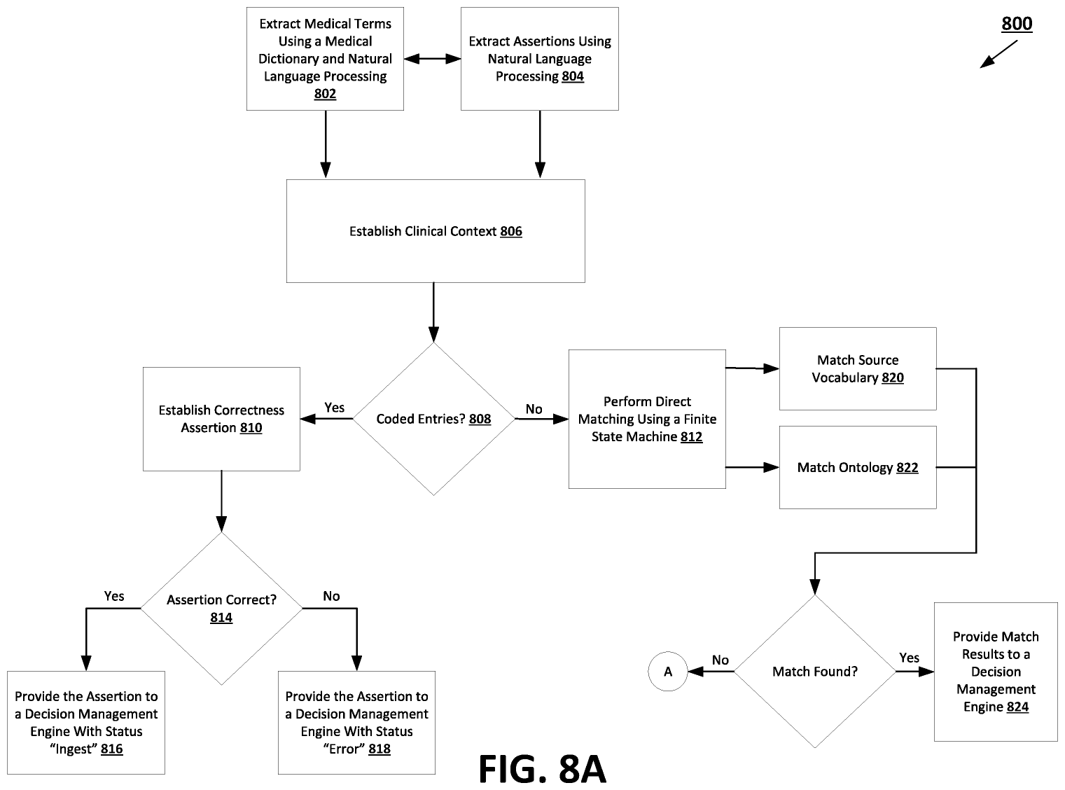

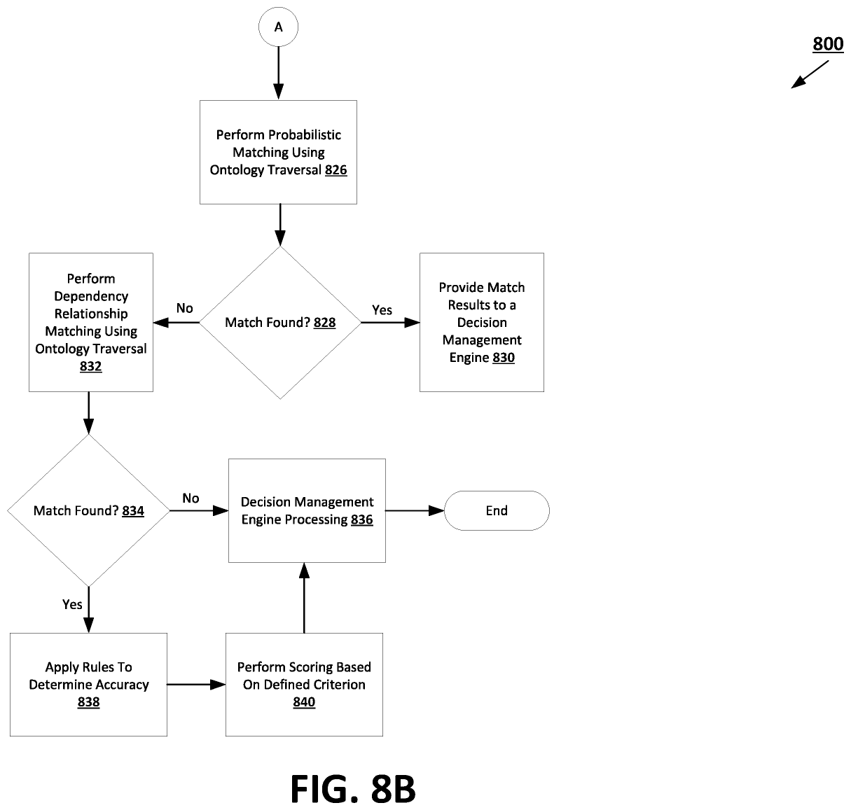

FIG. 8A illustrates an example system 800 for clinical data semantic interoperability using machine learning. In an embodiment, the system 800 can provide for consolidating and/or analyzing clinical data using machine learning, natural language processing and/or text analytics. The system 800 includes a step/operation 802 performed by the inference/rule engine 110 of the artificial intelligence computing entity 106 to extract medical terms using a medical dictionary and natural language processing. The system 800 includes a step/operation 804 performed by the inference/rule engine 110 of the artificial intelligence computing entity 106 to extract assertions using natural language processing. Furthermore, the system 800 includes a step/operation 806 performed by the inference/rule engine 110 of the artificial intelligence computing entity 106 to establish clinical context based at least in part on the step/operation 802 and/or the step/operation 804.

A step/operation 808 is also performed by the inference/rule engine 110 of the artificial intelligence computing entity 106 to determine whether there are coded entries (e.g., coded entries for the identified terms). If yes, a step/operation 810 is performed by the inference/rule engine 110 of the artificial intelligence computing entity 106 to establish correctness of the assertion. If no, a step/operation 812 is performed by the inference/rule engine 110 of the artificial intelligence computing entity 106 to perform direct matching using a finite state machine. After the step/operation 810 is performed, a step/operation 814 is performed by the inference/rule engine 110 of the artificial intelligence computing entity 106 to determine whether the assertion is correct. If yes, a step/operation 816 is performed by the inference/rule engine 110 of the artificial intelligence computing entity 106 to provide the assertion to a decision management engine (e.g., the decision management engine 112) with status “ingest.” If no, a step/operation 818 is performed by the inference/rule engine 110 of the artificial intelligence computing entity 106 to provide the assertion to a decision management engine (e.g., the decision management engine 112) with status “error.”

After the step/operation 812 is performed, a step/operation 820 is performed by the inference/rule engine 110 of the artificial intelligence computing entity 106 to match source vocabulary. Additionally, the step/operation 812 is performed, a step/operation 822 is performed by the inference/rule engine 110 of the artificial intelligence computing entity 106 to match ontology. A step/operation 808 is also performed by the inference/rule engine 110 of the artificial intelligence computing entity 106 to determine whether a match is found. If yes, a step/operation 824 is performed by the inference/rule engine 110 of the artificial intelligence computing entity 106 to provide a match to a decision management engine (e.g., the decision management engine 112).

If no, a step/operation 826 as shown in FIG. 8B is performed by the inference/rule engine 110 of the artificial intelligence computing entity 106 to perform probabilistic matching using ontology traversal. FIG. 8B further illustrates the example system 800 for clinical data semantic interoperability using machine learning. The system 800 includes a step/operation 828 performed by the inference/rule engine 110 of the artificial intelligence computing entity 106 to determine whether a match is found. If yes, a step/operation 830 is performed by the inference/rule engine 110 of the artificial intelligence computing entity 106 to provide a match to a decision management engine (e.g., the decision management engine 112). If no, a step/operation 832 is performed by the inference/rule engine 110 of the artificial intelligence computing entity 106 to perform dependency relationship matching using ontology traversal. The system 800 includes a step/operation 834 performed by the inference/rule engine 110 of the artificial intelligence computing entity 106 to determine whether a match is found. If no, a step/operation 836 is performed by the decision management engine 112 of the artificial intelligence computing entity 106 to facilitate decision management engine processing. If yes, a step/operation 838 is performed by the inference/rule engine 110 of the artificial intelligence computing entity 106 to apply rules to determine accuracy. Furthermore, step/operation 840 is performed by the inference/rule engine 110 of the artificial intelligence computing entity 106 to perform scoring based at least in part on defined criterion. Then, the step/operation 836 is performed by the decision management engine 112 of the artificial intelligence computing entity 106 to facilitate decision management engine processing.

### Clinical Data Semantic Interoperability Using Machine Learning

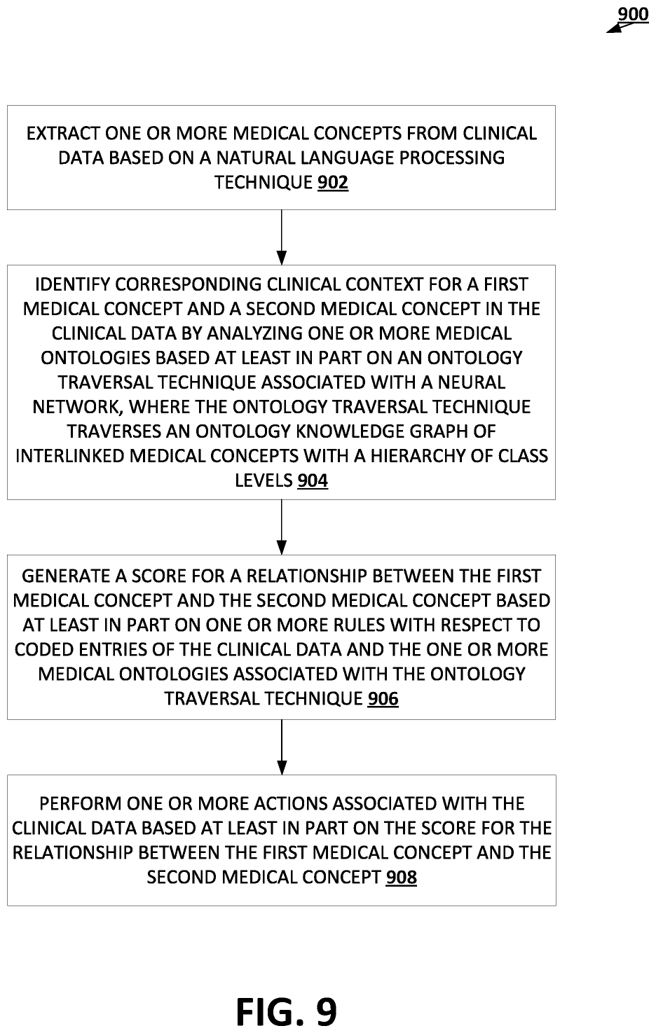

FIG. 9 is a flowchart diagram of an example process 900 for facilitating clinical data semantic interoperability using machine learning. Via the various steps/operations of process 900, the artificial intelligence computing entity 106 can process the clinical data 116 to provide improved clinical data and/or to initiate one or more workflows associated with the clinical data. In doing so, the artificial intelligence computing entity 106 can utilize machine learning solutions to infer important predictive insights and/or inferences from clinical data, for example.

The process 900 begins at step/operation 902 when the inference/rule engine 110 of the artificial intelligence computing entity 106 extracts one or more medical concepts from clinical data based at least in part on a natural language processing technique. In certain embodiments, the inference/rule engine 110 of the artificial intelligence computing entity 106 employs a medical dictionary and/or a medical thesaurus to extract one or more medical concepts from clinical data. In certain embodiments, the inference/rule engine 110 of the artificial intelligence computing entity 106 additionally or alternatively employs one or more statistical machine learning techniques to extract one or more medical concepts from clinical data.

At step/operation 904, the inference/rule engine 110 of the artificial intelligence computing entity 106 identifies corresponding clinical context for a first medical concept and a second medical concept in the clinical data by analyzing one or more medical ontologies based at least in part on an ontology traversal technique associated with a neural network. The ontology traversal technique traverses an ontology knowledge graph of interlinked medical concepts with a hierarchy of class levels. In certain embodiments, the inference/rule engine 110 of the artificial intelligence computing entity 106 traverses from a first class associated with a first hierarchy level of the ontology knowledge graph to a second class associated with a second hierarchy level of the ontology knowledge graph. In certain embodiments, the inference/rule engine 110 of the artificial intelligence computing entity 106 determines one or more relationships between the first medical concept and the second medical concept based at least in part on the ontology knowledge graph. In certain embodiments, the inference/rule engine 110 of the artificial intelligence computing entity 106 locates a parent concept associated with a medical concept in the ontology knowledge graph and/or stores data related to a list of child concepts associated with one or more related medical concepts.

At step/operation 906, the inference/rule engine 110 of the artificial intelligence computing entity 106 generates a score for a relationship between the first medical concept and the second medical concept based at least in part on one or more rules with respect to coded entries of the clinical data and the one or more medical ontologies associated with the ontology traversal technique. In certain embodiments, the inference/rule engine 110 of the artificial intelligence computing entity 106 determining the score based at least in part on one or more features included in patient data related to clinical data. Additionally or alternatively, the inference/rule engine 110 of the artificial intelligence computing entity 106 determining the score based at least in part on population data indicative of information related to one or more patient identities that are different than a patient identity associated with the clinical data.

At step/operation 908, the decision management engine 112 of the artificial intelligence computing entity 106 and/or the workflow management engine 114 of the artificial intelligence computing entity 106 performs one or more actions associated with the clinical data based at least in part on the score for the relationship between the first medical concept and the second medical concept. In certain embodiments, the decision management engine 112 of the artificial intelligence computing entity 106 and/or the workflow management engine 114 of the artificial intelligence computing entity 106 updates the clinical data based at least in part on the score for the relationship between the first medical concept and the second medical concept. Additionally or alternatively, the decision management engine 112 of the artificial intelligence computing entity 106 and/or the workflow management engine 114 of the artificial intelligence computing entity 106 invokes one or more workflows for the clinical data. Additionally or alternatively, the decision management engine 112 of the artificial intelligence computing entity 106 and/or the workflow management engine 114 of the artificial intelligence computing entity 106 renders data associated with the one or more medical ontologies via a graphical user interface of a computing device.

### V. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for facilitating clinical data semantic interoperability using machine learning, the computer-implemented method comprising:
extracting one or more medical concepts from clinical data based at least in part on a natural language processing technique;
identifying corresponding clinical context for a first medical concept and a second medical concept in the clinical data by analyzing one or more medical ontologies based at least in part on an ontology traversal technique associated with a neural network, wherein the ontology traversal technique traverses an ontology knowledge graph of interlinked medical concepts with a hierarchy of class levels;
generating a score for a relationship between the first medical concept and the second medical concept based at least in part on one or more rules with respect to coded entries of the clinical data and the one or more medical ontologies associated with the ontology traversal technique; and
performing one or more actions associated with the clinical data based at least in part on the score for the relationship between the first medical concept and the second medical concept.

2. The computer-implemented method of claim 1, wherein the identifying comprises traversing from a first class associated with a first hierarchy level of the ontology knowledge graph to a second class associated with a second hierarchy level of the ontology knowledge graph.

3. The computer-implemented method of claim 1, wherein the identifying comprises determining one or more relationships between the first medical concept and the second medical concept based at least in part on the ontology knowledge graph.

4. The computer-implemented method of claim 1, wherein the identifying comprises:
locating a parent concept associated with a medical concept in the ontology knowledge graph; and
storing data related to a list of child concepts associated with one or more related medical concepts.

5. The computer-implemented method of claim 1, wherein the generating the score comprises determining the score based at least in part on one or more features included in patient data related to clinical data.

6. The computer-implemented method of claim 1, wherein the generating the score comprises determining the score based at least in part on population data indicative of information related to one or more patient identities that are different than a patient identity associated with the clinical data.

7. The computer-implemented method of claim 1, wherein the performing the one or more actions comprises updating the clinical data based at least in part on the score for the relationship between the first medical concept and the second medical concept.

8. The computer-implemented method of claim 1, wherein the performing the one or more actions comprises invoking one or more workflows for the clinical data.

9. The computer-implemented method of claim 1, wherein the performing the one or more actions comprises rendering data associated with the one or more medical ontologies via a graphical user interface of a computing device.

10. An apparatus for facilitating recommendation prediction using machine learning, the apparatus comprising at least one processor and at least one memory including a computer program code, the at least one memory and the computer program code configured to, with the at least one processor, cause the apparatus to:
extract one or more medical concepts from clinical data based at least in part on a natural language processing technique;
identify corresponding clinical context for a first medical concept and a second medical concept in the clinical data by analyzing one or more medical ontologies based at least in part on an ontology traversal technique associated with a neural network, wherein the ontology traversal technique traverses an ontology knowledge graph of interlinked medical concepts with a hierarchy of class levels;
generate a score for a relationship between the first medical concept and the second medical concept based at least in part on one or more rules with respect to coded entries of the clinical data and the one or more medical ontologies associated with the ontology traversal technique; and
perform one or more actions associated with the clinical data based at least in part on the score for the relationship between the first medical concept and the second medical concept.

11. The apparatus of claim 10, wherein the at least one memory and the computer program code are configured to, with the at least one processor, cause the apparatus to:
traverse from a first class associated with a first hierarchy level of the ontology knowledge graph to a second class associated with a second hierarchy level of the ontology knowledge graph.

12. The apparatus of claim 10, wherein the at least one memory and the computer program code are configured to, with the at least one processor, cause the apparatus to:
determine one or more relationships between the first medical concept and the second medical concept based at least in part on the ontology knowledge graph.

13. The apparatus of claim 10, wherein the at least one memory and the computer program code are configured to, with the at least one processor, cause the apparatus to:
locate a parent concept associated with a medical concept in the ontology knowledge graph; and
store data related to a list of child concepts associated with one or more related medical concepts.

14. The apparatus of claim 9, wherein the score is generated based at least in part on one or more features included in patient data related to clinical data.

15. The apparatus of claim 9, wherein the score is generated based at least in part on population data indicative of information related to one or more patient identities that are different than a patient identity associated with the clinical data.

16. A non-transitory computer storage medium comprising instructions for facilitating recommendation prediction using machine learning, the instructions being configured to cause one or more processors to at least perform operations configured to:
extract one or more medical concepts from clinical data based at least in part on a natural language processing technique;
identify corresponding clinical context for a first medical concept and a second medical concept in the clinical data by analyzing one or more medical ontologies based at least in part on an ontology traversal technique associated with a neural network, wherein the ontology traversal technique traverses an ontology knowledge graph of interlinked medical concepts with a hierarchy of class levels;
generate a score for a relationship between the first medical concept and the second medical concept based at least in part on one or more rules with respect to coded entries of the clinical data and the one or more medical ontologies associated with the ontology traversal technique; and
perform one or more actions associated with the clinical data based at least in part on the score for the relationship between the first medical concept and the second medical concept.

17. The non-transitory computer storage medium of claim 16, wherein the operations are further configured to:
traverse from a first class associated with a first hierarchy level of the ontology knowledge graph to a second class associated with a second hierarchy level of the ontology knowledge graph.

18. The non-transitory computer storage medium of claim 16, wherein the operations are further configured to:
determine one or more relationships between the first medical concept and the second medical concept based at least in part on the ontology knowledge graph.

19. The non-transitory computer storage medium of claim 16, wherein the operations are further configured to:
locate a parent concept associated with a medical concept in the ontology knowledge graph; and
store data related to a list of child concepts associated with one or more related medical concepts.

20. The non-transitory computer storage medium of claim 16, wherein the score is generated based at least in part on population data indicative of information related to one or more patient identities that are different than a patient identity associated with the clinical data.
